(19) United States
(12) Patent Application Publication
(10) Pub. No.: US 2015/0292003 A1
(43) Pub. Date: Oct. 15, 2015

(54) METHODS AND KITS FOR DETECTION OF A PATHOGEN IN SUGARCANE

(71) Applicants: Chunyang FAN, Chapel Hill, NC (US); Wenjin YU, Cary, NC (US); Natassia CORREA, (US); Daniel Dias ROSA, Uberlandia, Minas Gerais (BR); Lambertus Pieter WOUDT, Grootebroek (NL); Manuel Benito SAINZ, San Paulo (BR); Kimberly Ann WHITE, (US); SYNGENTA PARTICIPATIONS AG, Basel (CH)

(72) Inventors: Chunyang Fan, Durham, NC (US); Wenjin Yu, Durham, NC (US); Natassia Correa, Parana (BR); Daniel Dias Rosa, Sao Paulo (BR); Lambertus Pieter Woudt, Enkhuizen (NL); Manuel Benito Sainz, Durham, NC (US); Kimberly Ann White, Durham, NC (US)

(73) Assignee: SYNGENTA PARTICIPATIONS AG, Basel (CH)

(21) Appl. No.: 14/437,664

(22) PCT Filed: Oct. 23, 2013
(86) PCT No.: PCT/US2013/066338

§ 371 Date: Apr. 22, 2015

Related U.S. Application Data
(60) Provisional application No. 61/717,908, filed on Oct. 24, 2012.

(51) Int. Cl. C12Q 1/68; C12Q 1/04

(52) U.S. Cl. CPC C12Q 1/689; C12Q 1/04

(57) ABSTRACT

Embodiments of the present invention provide a diagnostic approach utilizing quantitative polymerase chain reaction (PCR) to detect quantitatively a pathogen of the genus Leifsonia that causes ratoon stunting disease (RSD) in sugarcane. This is a rapid, cost-effective and/or high sensitivity methodology for detecting this pathogen. The present invention relates to methods and kits for detecting a pathogen in a plant, plant part or plant cell from the Gramineae/Poaceae family, such as plants of the Saccharum spp, including sugarcane.

### RELATED APPLICATION INFORMATION

This application claims the benefit of U.S. Provisional Application Ser. No. 61/717,908, filed Oct. 24, 2012, the disclosure of which is incorporated by reference herein in its entirety.

### STATEMENT REGARDING ELECTRONIC FILING OF A SEQUENCE LISTING

A Sequence Listing in ASCII text format, submitted under 37 C.F.R. §1.821, entitled 9207-90WO_{—} ST25.txt, 1,870 bytes in size, generated on Oct. 18, 2013 and filed via EFS-Web, is provided in lieu of a paper copy. This Sequence Listing is hereby incorporated by reference into the specification for its disclosures.

### FIELD OF THE INVENTION

The present invention relates to methods and kits for detecting a pathogen in a plant, plant part or plant cell from the Gramineae/Poaceae family, such as plants of the Saccharum spp, including sugarcane.

### BACKGROUND OF THE INVENTION

Leifsonia xyli subsp. xyli (Lxx) are gram-positive, nutritional fastidious bacteria. Lxx grow very slowly and can be difficult to culture. It generally takes about four weeks to obtain a visible single colony, if cultured at 28° C. Lxx is one of the most economically important pathogens in sugarcane worldwide. The Lxx pathogen causes ratoon stunting disease (RSD) in sugarcane. Although this disease does not have reliable external symptoms, it may reduce sugarcane yield up to 30% in some susceptible varieties. RSD is typically transferred from infected canes through mechanical harvest (1, 2).

Three methodologies are currently employed to detect the Lxx pathogen (3-5): serological dot blotting; microscopy; and conventional polymerase chain reaction (PCR). However, the limit of detection present in serological assays is only about 100,000 copies/mL, and this method requires sugarcane juice that has been concentrated as well as high titer antibodies. Lxx detection with microscopy is a low throughput and labor intensive method. Lastly, while PCR methods have been reported to detect Lxx, these protocols are generally based on conventional PCR amplification methods. These conventional PCR-based methods are only qualitative approaches merely detecting the presence of Lxx without quantitation, and these methods have also proven to be less sensitive than the serological approaches presently available.

### SUMMARY OF THE INVENTION

Aspects of the present invention provide a quantitative approach for the detection of Lxx in sugarcane. More specifically, aspects of the present invention provide a quantitative and/or sensitive approach based on real-time PCR (also called quantitative real-time PCR or quantitative PCR) for detection of the Lxx pathogen in sugarcane juice.

Further aspects of the present invention provide oligonucleotide primers for use in a quantitative amplification-based detection of a nucleic acid sequence from the Lxx pathogen, wherein said primers comprise, consist essentially of and/or consist of a sequence having sequence identity with at least 10 contiguous nucleotides of a nucleic acid sequence from (a) 16S-23S ribosomal RNA intergenic transcribed spacer (ITS) in the Lxx genome (3), or (b) a nucleic acid sequence from ISLxx4 that encodes the tnp transposase from Lxx (6).

Aspects of the invention also provide methods for the detection of a microorganism belonging to the genus Leifsonia, comprising subjecting a sugarcane sample to quantitative polymerase chain reaction amplification using a pair of oligonucleotide primers as described herein, and detecting Leifsonia by visualizing the product of the quantitative polymerase chain reaction amplification. In particular aspects, the amplification process includes amplifying at least a part of the Leifsonia xyli subsp. xyli intergenic transcribed spacer sequence. In other aspects, the amplification process includes amplifying at least a part of a nucleic acid sequence from ISLxx4 that encodes the tnp transposase from Lxx.

Still further aspects of the present invention provide diagnostic kits used in detecting a microorganism belonging to the genus Leifsonia, comprising the oligonucleotide primers described herein. In particular embodiments, the diagnostic kits also include a labeled reporter probe for use in detecting the microorganism belonging to the genus Leifsonia. In further embodiments, the probe is a fluorescently labeled probe.

Aspects of the present invention also provide methods for preparing a sample from a sugarcane plant for detecting a microorganism, comprising diluting the sample at least five-fold in an aqueous medium.

The approach embodied in aspects of the present invention can eliminate the costly and time-consuming steps of DNA isolation or pathogen lysis as described in other published methods for the detection of the Lxx pathogen. The limit of detection (LOD) for the diagnostic approach according to embodiments of the present invention is between about 4,000 and 9,000 copies of Lxx pathogen/mL, and in some cases, about 4,000 copies of Lxx pathogen/mL. The diagnostic methods described herein may be used to detect the presence of ratoon stunting disease in sugarcane, in the field or commercial sugarcane, as well as provide utility in quality control in sugarcane nurseries in order to assist sugarcane breeders in delivering certified RSD-free sugarcane seeds and/or RSD-free stem cuttings.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### BRIEF DESCRIPTION OF THE DRAWINGS

FIGS. 1A and 1B depict the BLAST analysis of the two PCR amplicons from (A) Assay-1 and (B) Assay-2 produced using PCR primers according to embodiments of the invention.

FIG. 2 depicts a standard curve generated from plotting Ct value against Lxx copy number determined through methods of the present invention used in quantitation of Lxx copy number in a sugarcane juice sample.

### DETAILED DESCRIPTION

Embodiments of the present invention are explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which does not depart from the instant invention. Hence, the following specification is intended to illustrate some particular embodiments of the invention, and not to exhaustively specify all permutations, combinations and variations thereof.

Unless otherwise defined, all technical and scientific teens used herein have the same meaning as commonly understood by one of ordinary skill in the art to which this invention belongs. The terminology used in the description of the invention herein is for the purpose of describing particular embodiments only and is not intended to be limiting of the invention.

As used in the description of the embodiments of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, ±5%, ±1%, ±0.5%, or even ±0.1% of the specified amount.

Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

The term “consists essentially of” and grammatical variants), as applied to a polynucleotide sequence of this invention, means a polynucleotide that consists of both the recited sequence (e.g., SEQ ID NO:) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered from the requirements for detection of the Lxx pathogen as set forth herein.

Nucleotide sequences are presented herein by single strand only, in the 5′ to 3′ direction, from left to right, unless specifically indicated otherwise. Any nucleotides represented herein in the manner recommended by the IUPAC-IUB Biochemical Nomenclature Commission in accordance with 37 C.F.R. §1.822 and established usage.

As used herein, “nucleic acid,” “nucleotide sequence,” “oligonucleotide” and “polynucleotide” are used interchangeably and encompass both RNA and DNA, including cDNA, genomic DNA, mRNA, synthetic (e.g., chemically synthesized) DNA or RNA and chimeras of RNA and DNA. The term oligonucleotide, polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention.

The nucleic acids, oligonucleotides and polynucleotides of the invention can be isolated. An “isolated” nucleic acid molecule, oligonucleotide or polynucleotide is a nucleic acid molecule, oligonucleotide or polynucleotide that, by the hand of man, exists apart from its native environment and is therefore not a product of nature. An isolated nucleic acid molecule, isolated oligonucleotide or isolated polynucleotide may exist in a purified form or may exist in a non-native environment such as, for example, a recombinant host cell. Thus, for example, the term “isolated” means that it is separated from the chromosome and/or cell in which it naturally occurs. A nucleic acid, oligonucleotide or polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules, oligonucleotide and polynucleotides of the invention can be considered to be “isolated.”

Further, an “isolated” nucleic acid, oligonucleotide or polynucleotide can be a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. The “isolated” nucleic acid, oligonucleotide or polynucleotide can exist in a cell (e.g., a plant cell), optionally stably incorporated into the genome. According to this embodiment, the “isolated” nucleic acid, oligonucleotide or polynucleotide can be foreign to the cell/organism into which it is introduced, or it can be native to an the cell/organism, but exist in a recombinant form (e.g., as a chimeric nucleic acid or polynucleotide) and/or can be an additional copy of an endogenous nucleic acid or polynucleotide. Thus, an “isolated nucleic acid molecule” “isolated oligonucleotide” or “isolated polynucleotide” can also include a nucleotide sequence derived from and inserted into the same natural, original cell type, but which is present in a non-natural state, e.g., present in a different copy number, in a different genetic context and/or under the control of different regulatory sequences than that found in the native state of the nucleic acid molecule or polynucleotide.

In representative embodiments, the “isolated” nucleic acid, oligonucleotide or polynucleotide is substantially free of cellular material (including naturally associated proteins such as histones, transcription factors, and the like), viral material, and/or culture medium (when produced by recombinant DNA techniques), or chemical precursors or other chemicals (when chemically synthesized). Optionally, in representative embodiments, the isolated nucleic acid, oligonucleotide or polynucleotide is at least about 10%, 20%, 30%, 40%, 50%, 60%, 70%, 80%, 90%, 95% or more pure.

As used herein, the term “recombinant” nucleic acid, oligonucleotide, polynucleotide or nucleotide sequence refers to a nucleic acid, polynucleotide or nucleotide sequence that has been constructed, altered, rearranged and/or modified by genetic engineering techniques. The term “recombinant” does not refer to alterations that result from naturally occurring events, such as spontaneous mutations, or from non-spontaneous mutagenesis.

The term “fragment,” “portion,” “part” as applied to a nucleic acid sequence, oligonucleotide or polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 5, 8, 10, 12, 15, 20, 25, 30, 35, 40, 45, 50, 60, 70 or more consecutive nucleotides of a nucleic acid or nucleotide sequence according to the invention.

A “biologically active” nucleotide sequence is one that substantially retains at least one biological activity normally associated with the wild-type nucleotide sequence, for example, promoter activity, optionally inducible promoter activity in response to exposure to nitrate, drought or rehydration. In particular embodiments, the “biologically active” nucleotide sequence substantially retains all of the biological activities possessed by the unmodified sequence. By “substantially retains” biological activity, it is meant that the nucleotide sequence retains at least about 50%, 60%, 75%, 85%, 90%, 95%, 97%, 98%, 99%, or more, of the biological activity of the native nucleotide sequence (and can even have a higher level of activity than the native nucleotide sequence). Methods of measuring promoter activity are known in the art.

Two nucleotide sequences are said to be “substantially identical” to each other when they share at least about 60%, 70%, 75%, 80%, 85%, 90%, 95%, 97%, 98%, 99% or even 100% sequence identity. In some particular embodiments, the nucleotide sequences of the present invention include nucleotides sequences having 90%, 95%, 97%, 98%, or 99% sequence identity to the nucleotide sequences of the invention.

Two amino acid sequences are said to be “substantially identical” or “substantially similar” to each other when they share at least about 60%, 70%, 75%, 80%, 85%, 90%, 95%, 97%, 98%, 99% or even 100% sequence identity or similarity, respectively.

As used herein “sequence identity” refers to the extent to which two optimally aligned polynucleotide or polypeptide sequences are invariant throughout a window of alignment of components, e.g., nucleotides or amino acids.

As used herein “sequence similarity” is similar to sequence identity (as described herein), but permits the substitution of conserved amino acids (e.g., amino acids whose side chains have similar structural and/or biochemical properties), which are well-known in the art.

As is known in the art, a number of different programs can be used to identify whether a nucleic acid has sequence identity or an amino acid sequence has sequence identity or similarity to a known sequence. Sequence identity or similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, PASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp, CABIOS 5, 151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al, Nucleic Acids Res. 25, 3389-3402 (1997).

The CLUSTAL program can also be used to determine sequence similarity. This algorithm is described by Higgins et al. (1988) Gene 73:237; Higgins et al. (1989) CABIOS 5:151-153; Carpet et al. (1988) Nucleic Acids Res. 16: 10881-90; Huang et al. (1992) CABIOS 8: 155-65; and Pearson et al. (1994) Meth. Mol. Biol. 24: 307-331.

The alignment may include the introduction of gaps in the sequences to be aligned. In addition, for sequences which contain either more or fewer nucleotides than the nucleic acids disclosed herein, it is understood that in one embodiment, the percentage of sequence identity will be determined based on the number of identical nucleotides acids in relation to the total number of nucleotide bases. Thus, for example, sequence identity of sequences shorter than a sequence specifically disclosed herein, will be determined using the number of nucleotide bases in the shorter sequence, in one embodiment. In percent identity calculations relative weight is not assigned to various manifestations of sequence variation, such as, insertions, deletions, substitutions, etc.

Two nucleotide sequences can also be considered to be substantially identical when the two sequences hybridize to each other under stringent conditions. A non-limiting example of “stringent” hybridization conditions include conditions represented by a wash stringency of 50% formamide with 5×Denhardt's solution, 0.5% SDS and 1×SSPE at 42° C. “Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). In some representative embodiments, two nucleotide sequences considered to be substantially identical hybridize to each other under highly stringent conditions. Generally, highly stringent hybridization and wash conditions are selected to be about 5° C. lower than the thermal melting point (Tₘ) for the specific sequence at a defined ionic strength and pH.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

An “isolated” polypeptide is a polypeptide that, by the hand of man, exists apart from its native environment and is therefore not a product of nature. An isolated polypeptide may exist in a purified form or may exist in a non-native environment such as, for example, a recombinant host cell.

In representative embodiments, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. In particular embodiments, the “isolated” polypeptide is at least about 1%, 5%, 10%, 25%, 50%, 60%, 70%, 75%, 80%, 85%, 90%, 95%, 97%, 98%, 99% or more pure (w/w). In other embodiments, an “isolated” polypeptide indicates that at least about a 5-fold, 10-fold, 25-fold, 100-fold, 1000-fold, 10,000-fold, or more enrichment of the protein (w/w) is achieved as compared with the starting material.

A “biologically active” polypeptide is one that substantially retains at least one biological activity normally associated with the wild-type polypeptide. In particular embodiments, the “biologically active” polypeptide substantially retains all of the biological activities possessed by the unmodified (e.g., native) sequence. By “substantially retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide).

As used herein, in some embodiments, “plant,” “plant part,” “plant tissue” are used interchangeably, and for use in the methods of the invention means plant organs (e.g., leaves, stems, shoots, roots, etc.), seeds, plant cells, and progeny of the same. Thus, plant, plant part, plant tissue also includes, without limitation, protoplasts, nodules, nodes, callus (e.g., embryogenic callus tissue), suspension culture, embryos, as well as flowers, ovules, stems, fruits, leaves, side shoots (also referred to as tillers), roots, root tips and the like originating in plants or their progeny. Plant cell includes, without limitation, a cell obtained from a seed, embryo, meristematic region, callus tissue, suspension culture, leaf, primary stalk, root, shoot, gametophyte, sporophyte, pollen and/or microspore. Accordingly, a plant, plant part, plant tissue includes, but is not limited, to reproductive tissues (e.g., petals, sepals, stamens, pistils, receptacles, anthers, pollen, flowers, fruits, flower bud, ovules, seeds, embryos); vegetative tissues (e.g., petioles, stems, roots, root hairs, root tips, pith, coleoptiles, stalks, shoots, branches, bark, apical meristem, axillary bud, cotyledon, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs plant cell tissue cultures, plant calli, plant clumps, and the like.

As used herein, “side shoot” means a shoot other than the primary shoot (i.e., stalk) originating from the crown of the sugar cane plant close to the soil surface. A side shoot may also be referred to as a “secondary shoot”.

The term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ.

Plants employed in practicing the present invention include those belonging to the Gramineae/Poaceae family, and in particular, plants belonging to the genus Saccharum, such as sugarcane as understood by one of skill in the art. Exemplary sugarcane species include, but are not limited to, Saccharum arundinaceum, Saccharum barberi, Saccharum bengalense, Saccharum edule, Saccharum munja, Saccharum officinarum, Saccharum procerum, Saccharum ravennae, Saccharum robustum, Saccharum sinense, Saccharum spontaneum and cultivated and/or hybrid species thereof.

The plant, plant part and plant tissue of the present invention can be derived from greenhouse grown plants or from field grown plants. As used herein, an “open system” refers to outdoors, for example, field crops, landscape settings, harvested plants or in a cargo unit for transport of the plants, wherein the plants are substantially exposed to the outdoors. As further used herein, a “closed system” refers to indoors, for example, a greenhouse, a warehouse, a plant in a container housed indoors or inside a cargo unit for transport of the plants, wherein the plants are not substantially exposed to the outdoors.

As used herein, a “sample” refers to a plant or substance obtained therefrom. With respect to sugarcane, the sample may be the juice extracted from a sugarcane plant. The sample may be obtained from the plant by extracting juice from a plant part, for example, the vascular tissues such as the xylem. Extraction may occur by crushing a plant part. For example, juice may be extracted, i.e., removed from a sugar cane node. The term “node” means the part of the stem of a plant from which a leaf, branch, or aerial root grows; each plant has many nodes. In particular embodiments, sugarcane culms may be cleaned to avoid contamination by soil particles and other impurities and stems may be cut between two neighboring internodes on order to obtain a sample.

The genus Leifsonia was originally described by Evtushenko et al. Int J Syst Evol Microbiol. (2000) January; 50 Pt 1:371-80 and includes bacteria that are gram-positive, non-spore-forming, rod-shaped (or filamentous), obligately aerobic and/or catalase-positive. Species include, but are not limited to, Leifsonia antarctica, Leifsonia aquatica, Leifsonia kafniensis, Leifsonia naganoensis, Leifsonia pindariensis, Leifsonia kribbensis, Leifsonia lichenia, Leifsonia sp. 1.5-VEs, Leifsonia poae, Leifsonia rubra, Leifsonia shinshuensis, Leifsonia xyli, Leifsonia sp. 1c, Leifsonia sp. 1Ucecto113, Leifsonia sp. 24, Leifsonia sp. 3030, Leifsonia sp. 31ND1, Leifsonia sp. 3_{—}1K, Leifsonia sp. 1.7-05, Leifsonia sp. 1012, Leifsonia sp. 1019, Leifsonia sp. 1022, Leifsonia sp. 4-8, Leifsonia sp. 5002, Leifsonia sp. 5011, Leifsonia sp. 5012, Leifsonia sp. 555-1, Leifsonia sp. 5GH 26-15, Leifsonia sp. 5GHs34-4, Leifsonia sp. 6002, Leifsonia sp. 6003, Leifsonia sp. 7PE5.1, Leifsonia sp. 7PE5.6, Leifsonia sp. 7PE5.9, Leifsonia sp. 8_{—}1Ka, Leifsonia sp. A19MG2, Leifsonia sp. 4-16, Leifsonia sp. 4-69, Leifsonia sp. AaD62c, Leifsonia sp. AaM53a, Leifsonia sp. AaM56a, Leifsonia sp. AaM61b, Leifsonia sp. AaM67a, Leifsonia sp. AaM69a, Leifsonia sp. ACT1EB, Leifsonia sp. AH86, Leifsonia sp. AK43 3.1, Leifsonia sp. ARS-50, Leifsonia sp. ASS1, Leifsonia sp. ATSB20, Leifsonia sp. ATSB24, Leifsonia sp. AW16, Leifsonia sp. B-G-NA5, Leifsonia sp. BF55, Leifsonia sp. C13X, Leifsonia sp. C14, Leifsonia sp. A5AG1, Leifsonia sp. A5AGK, Leifsonia sp. A5ATF, Leifsonia sp. A6-1, Leifsonia sp. A6ACH, Leifsonia sp. A6ATA, Leifsonia sp. AaD57b, Leifsonia sp. JC2485, Leifsonia sp. JDM-3-01, Leifsonia sp. JDM301, Leifsonia sp. KACC 13362, Leifsonia sp. KNF2, Leifsonia sp. L1907, Leifsonia sp. L89, Leifsonia sp. LAA-2009-i47, Leifsonia sp. C6, Leifsonia sp. CHNTR46, Leifsonia sp. CHNTR47, Leifsonia sp. CJ-G-R2A10, Leifsonia sp. CJ-G-R2A8, Leifsonia sp. CK32 7.1, Leifsonia sp. CL33 6.2, Leifsonia sp. COL-14, Leifsonia sp. CYEB-21, Leifsonia sp. D69, Leifsonia sp. DAB_ATA115, Leifsonia sp. DAB_ATA119, Leifsonia sp. DAB_MOR27, Leifsonia sp. DAB_MOR44, Leifsonia sp. DAB_ST76, Leifsonia sp. DAB_ST77, Leifsonia sp. DAB_ST78, Leifsonia sp. DAB_ST80, Leifsonia sp. DAB_ST84, Leifsonia sp. DAB_ST85, Leifsonia sp. DAB_ST88, Leifsonia sp. DAB_ST90, Leifsonia sp. Ellin419, Leifsonia sp. Ellin432, Leifsonia sp. FS-YC6687, Leifsonia sp. FS-YC6688, Leifsonia sp. FS14-4, Leifsonia sp. HPABA04, Leifsonia sp. i1(2010), Leifsonia sp. i4(2010), Leifsonia sp. ice-oil-482, Leifsonia sp. IMCr08, Leifsonia sp. IMER-B2-13, Leifsonia sp. INBio2556G, Leifsonia sp. PDD-32b-20, Leifsonia sp. PTX1, Leifsonia sp. Q1, Leifsonia sp. Q6, Leifsonia sp. qy20, Leifsonia sp. R-45745, Leifsonia sp. R-46062, Leifsonia sp. R-46076, Leifsonia sp, R-46167, Leifsonia sp. R-46259, Leifsonia sp. RB-62, Leifsonia sp. RNE 15, Leifsonia sp. RODXS12, Leifsonia sp. RODXS16, Leifsonia sp. RR6, Leifsonia sp. RU-20, Leifsonia sp. RX68, Leifsonia sp. S1.ACT003, Leifsonia sp. S14-15, Leifsonia sp. S2-2, Leifsonia sp. S2.ACT.008, Leifsonia sp. S24526, Leifsonia sp. S3.TSA.014, Leifsonia sp. S3H4, Leifsonia sp. S4-9, Leifsonia sp. S717-51, Leifsonia sp. S749, Leifsonia sp. SAP34_{—}5, Leifsonia sp. SAP37_{—}3, Leifsonia sp. SAP60_{—}1, Leifsonia sp. SaPR11, Leifsonia sp. SaPS2, Leifsonia sp. SaZR8, Leifsonia sp. LI1, Leifsonia sp. M-BtII-1, Leifsonia sp. M060706-2, Leifsonia sp. M060706-3, Leifsonia sp. M060706-4, Leifsonia sp. m9-17, Leifsonia sp. m9-18, Leifsonia sp. m9-51, Leifsonia sp. mat852, Leifsonia sp, mat858, Leifsonia sp. M115-7, Leifsonia sp. MM91(2011), Leifsonia sp. MMA-A-1, Leifsonia sp. MMA-A1-3, Leifsonia sp. MMA-BI-2, Leifsonia sp. MMA-C-1, Leifsonia sp. MN 177, Leifsonia sp. MN10-1, Leifsonia sp. MN10-2, Leifsonia sp. MN6-24, Leifsonia sp. MN6-25, Leifsonia sp. MSL 02, Leifsonia sp. MSL 07, Leifsonia sp. MSL 27, Leifsonia sp. NaF-A-1, Leifsonia sp. NaF-BI-2, Leifsonia sp. NaF-BtI-2, Leifsonia sp. Leifsonia sp. ODP61203by2, Leifsonia sp. OKI-36, Leifsonia sp. OK1-4, Leifsonia sp. P1, Leifsonia sp. PC-07, Leifsonia sp. PDD-23b-16, Leifsonia sp. TG-S240, Leifsonia sp. TG-S248, Leifsonia sp. Tianshan517-2, Leifsonia sp. TP1MI, Leifsonia sp. TP2ME, Leifsonia sp. TSA62, Leifsonia sp. TSA71, Leifsonia sp. UFLA04-285, Leifsonia sp. UST050418-516, Leifsonia sp. V4.MO.14, Leifsonia sp. W3, Leifsonia sp. wged116, Leifsonia sp. wged95, Leifsonia sp. WPCB149, Leifsonia sp. YIM C813, Leifsonia sp. YN-19, Leifsonia sp. SBI-1, Leifsonia sp. SBI-2, Leifsonia sp. SBI-3, Leifsonia sp. SMCC 80627, Leifsonia sp. SMCC B0673, Leifsonia sp. swb-15, Leifsonia sp. Z3-YC6862, Leifsonia sp. ZS2-1, Leifsonia sp. ZS3-10-1, Leifsonia sp. ZS5-26, Leifsonia sp. ZS5-27, Leifsonia sp. ZS5-15 and Leifsonia sp. ZS5-30. See, for example, NCBI taxonomy database. In particular embodiments, the microorganism belongs to the species Leifsonia xyli and subspecies, Leifsonia xyli subsp. xyli. (Lxx).

Except as otherwise indicated, standard molecular biological methods known to those skilled in the art may be used for amplifying, analyzing, detecting, isolating and identifying nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

Embodiments of the present invention provide methods for detection of or assay for a microorganism belonging to the genus Leifsonia in plants. More particularly, the detection method of the invention comprises amplification of a nucleic acid sequence from a sample from a sugarcane plant, such as sugarcane juice, wherein the nucleic acid sequence to be amplified is an Lxx nucleic acid sequence, and detection of specific amplification of said nucleic acid sequence is indicative of the presence of the Lxx pathogen in sugarcane.

In some embodiments of the invention, the nucleic acid sequence amplified is a sequence from the 16S-23S ribosomal RNA intergenic transcribed spacer (ITS) in the Lxx genome (GenBank Accession No. EU723209). In a more particular embodiment, the nucleic acid sequence amplified from the 16S-23S ribosomal RNA ITS in the Lxx genome comprises, consists essentially of and/or consists of the sequence CGCCGGATCTGAGACAGTACTTATCACATCGGTACGACTGGGTCTCAGCCGGTCA GCTCATGGGTGGAACATTGACAT (SEQ ID NO:1).

According to further embodiments, the invention comprises primers for amplification of the sequence set forth in SEQ ID NO:1. In particular embodiments, the sequence of the primers comprise, consist essentially of and/or consist of the sequences CGCCGGATCTGAGACAGTACT (SEQ ID NO:2) and/or ATGTCAATGTTCCACCCATGAG (SEQ ID NO:3), or a sequence having sequence identity with at least 10 contiguous nucleotides thereof.

In yet another embodiment, the nucleic acid sequence amplified is from ISLxx4 in the Lxx genome (NCBI Accession No. NC_{—}006087). In a more particular embodiment, the nucleic acid sequence amplified from ISLxx4 in the Lxx genome comprises, consists essentially of and/or consists of the sequence CCTTGCCAGGCTCATCGTCGAGGACCATTGGCTGGTCTCCGTCGCAGCGAAGATG TTTATGGTCTCGCCCG (SEQ ID NO:4).

In still further embodiments, the invention comprises primers for amplification of the sequence as set forth in SEQ ID NO:4. In particular embodiments, the primers for amplification of this sequence comprise, consist essentially of and/or consist of the sequences CCTTGCCAGGCTCATCGT (SEQ ID NO:5) and CGGGCGAGACCATAAACATC (SEQ ID NO:6), or a sequence having sequence identity with at least 10 contiguous nucleotides thereof.

In another embodiment, the nucleic acid amplification method of the invention is utilized to quantitatively determine or assay for the presence of the Lxx pathogen in sugarcane. Such quantitative or real-time amplification, for example, quantitative or real-time polymerase chain reaction (qPCR), may be performed using any protocol and instrumentation for quantitative real-time amplification available and within the understanding of one of skill in the art.

In a further embodiment, the protocol used for the quantitative or real-time amplification is probe-based, and comprises the use of a fluorescently labeled or fluorogenic nucleic acid reporter probe that is specific to the amplified nucleic acid sequence. The fluorescent label of the reporter probe may be any label that is available and/or known by one of skill in the art. In a particular embodiment, the fluorescently labeled or fluorogenic nucleic acid reporter probe comprises a fluorescent label and a quenching moiety, wherein the reporter probe does not fluoresce until it is broken down by the amplification reaction, when the fluorescent label is separated from the quenching moiety. Thus, the amplification product may be visualized and further quantified through the detection of fluorescence. The detection of fluorescence from the fluorescent label may be performed using any protocol or instrumentation for the detection of fluorescence that would be known and within the purview of one of skill in the art to quantitatively analyze the amplification product from a sample. Through the quantitation of the amplification product, both the presence and the extent of the presence of the Lxx pathogen may be determined from a sample of, for example, sugarcane.

In a more particular embodiment, the fluorescent probe comprises a carboxyfluorescein (PAM) label. In a further particular embodiment, the PAM label is located at the 5′ end of the fluorescent report probe. The fluorogenic nucleic acid report probe of the invention for use in quantitative or real-time amplification may comprise a quenching moiety, which quenches the fluorescence of the label on the probe. The quenching moiety may be any quenching moiety that is available and/or known by one of skill in the art. In a particular embodiment, the quenching moiety may be BLACK HOLE QUENCHER-1® (BHQ-1). In a further particular embodiment, the BHQ-1 quenching moiety is located at the 3′ end of the fluorescent reporter probe. In a further embodiment, the quantitative real-time amplification, detection and quantitation protocol used is the commercially available TAQMAN® protocol.

In another particular embodiment, the invention comprises a probe for the nucleic acid sequence amplified from the 16S-23S ribosomal RNA ITS in the Lxx genome. In a more particular embodiment, the probe sequence comprises, consists essentially of and/or consists of the sequence TCACATCGGTACGACTGGGTCTCAGC (SEQ ID NO:7), or a sequence having sequence identity with at least 10 contiguous nucleotides thereof.

In yet another particular embodiment, the invention comprises a probe for the nucleic acid sequence amplified from ISLxx4 in the Lxx genome. In another more particular embodiment, the probe sequence comprises, consists essentially of and/or consists of the sequence ATTGGCTGGTCTCCGTCGCAGC (SEQ ID NO:8), or a sequence having sequence identity with at least 10 contiguous nucleotides thereof.

In further embodiments of the present invention, in the methods of detecting the microorganism or assaying for detection of the same, the sample is diluted prior to being subjected to the quantitative PCR amplification process. In some embodiments, the sample can be diluted at least two, three, four or five fold. The dilution may be a serial dilution or a volume to volume dilution. In particular embodiments, the dilution is a five-fold dilution.

Further, the methods of detecting the microorganism or assaying for the detection of the same can be conducted in an open system such as outdoors, for example, in a field grown as a crop, in a landscape environment or in an outdoor environment containing harvested plants. Alternatively or additionally, the methods of detecting the microorganism or assaying for the detection of the same can be conducted in a closed system such as indoors, for example, a greenhouse, a warehouse, a plant in a container housed indoors, an indoor environment containing harvested plants or inside a cargo unit for transport of the plants, wherein the plants are not substantially exposed to the outdoors.

In addition to the nucleic acid primers and probes as set forth above, further embodiments of the invention may also comprise a kit for the detection of the Lxx pathogen in a sample. In a particular embodiment, the kit may comprise the primers having a sequence as set forth in SEQ ID NOs:2 and 3, or a fragment of at least 10 contiguous nucleotides thereof, and the fluorescent reporter probe having a sequence as set forth in SEQ ID NO:7, or a sequence having sequence identity with at least 10 contiguous nucleotides thereof. In another particular embodiment, the diagnostic kit may comprise the primers having a sequence as set forth in SEQ ID NOs:5 and 6, or a fragment of at least 10 contiguous nucleotides thereof, and the fluorescent reporter probe having a sequence as set forth in SEQ ID NO:8, or a sequence having sequence identity with at least 10 contiguous nucleotides thereof.

The kits further include the elements necessary to carry out the process described above. Such a kit may comprise a carrier being compartmentalized to receive in close confinement therein one or more container, such as tubes or vials. One of the containers may contain unlabeled or detectably labeled primers. The labeled primers may be present in lyophilized form or in an appropriate buffer as necessary. One or more containers may contain one or more enzymes or reagents to be utilized in the PCR reactions. These enzymes may be present by themselves or in admixtures, in lyophilized form or in appropriate buffers. The kit may contain all of the additional elements necessary to carry out techniques of the invention, such as buffers, extraction reagents, enzymes, pipettes, plates, nucleic acids, nucleoside triphosphates, filter paper, gel materials, transfer materials, autoradiography supplies, instructions and the like.

Embodiments of the present invention further provide methods for preparing a sample from a sugarcane plant for detecting a microorganism. The method comprises, in particular, diluting the sample in an aqueous medium. The aqueous medium can be water. The sample can be diluted at least two, three, four or five fold. The dilution may be a serial dilution or a volume to volume dilution. In particular embodiments, the dilution is a five-fold dilution. In particular embodiments, the microorganism is Leifsonia xyli subsp. xyli. The microorganism is detected using quantitative polymerase chain reaction. The process of preparing the sample, which can be used to detect or assay for the microorganism as described herein, can be carried out in an open system or a closed system as described herein. The actual detection or assay step can be carried out within seconds, minutes, days, hours, months or years of preparing the sample for the detection or assay step. Embodiments of the present invention do not include a nucleic acid isolation or pathogen lysis step as described in conventional methods of preparing a sample for detection of the microorganism described herein.

Embodiments of the present invention further provide a quality control method for sugarcane breeders and/or inspectors for the detection of the presence of RSD in commercial sugarcane comprising utilizing the methods described herein for the detection of the microorganisms described herein using a quantitative polymerase chain reaction amplification process. The detection of the amplification product is indicative of the presence of RSD.

Embodiments of the present invention also provide a quality control method for sugarcane breeders for the production of RSD-free sugarcane stem cuttings and/or RSD-free sugarcane seeds comprising utilizing the methods described herein for the detection of the microorganisms described herein and obtaining seeds only from those plants that test negative for RSD, i.e., the microorganism associated with RSD is not detected using a quantitative polymerase chain reaction amplification process evidenced by the lack of significant detection of the amplification product.

The present invention is more particularly described in the following examples that are intended as illustrative only since numerous modifications and variations therein will be apparent to those skilled in the art.

### Example

### Material and Methods

Samples:

Sugarcane juices from Lxx infected and Lxx-free sugarcane. The sugarcane culms were cleaned with a damp cloth to avoid contamination by soil particles and other impurities. Stems were cut between two neighboring internodes. A low pressure compressor was used in extraction of the sugarcane juice. About 0.5 mL juice from each stem was collected into a 1.5 mL microcentrifuge tube.

DNA Template Preparation for TAQMAN® Assays:

compared to published methods of detection of Lxx, there is no pathogen lysis step in this method. The original sugarcane juice is diluted 5-fold in water or Tris and EDTA (TE) buffer. The sugarcane juice can be diluted from 5, 6, 7, 8, 9, or 10-fold in water or TE buffer. The DNA of the pathogen of interest will be released during the first denaturation step (about 95° C., 5 min) in a real-time PCR reaction. This method simplifies the detection process.

TAQMAN® Assay Design:

Two TAQMAN® assays were designed using Primer Express 3.0 (Applied Biosystems, Inc.). Assay-1 was designed based on a DNA sequence from the 16S-23S ribosomal RNA intergenic transcribed spacer (ITS) in the Lxx genome and Assay-2 was designed based on a sequence from ISLxx4 that encodes the tnp transposase from Lxx. There are 26 copies of ISLxx4 in the Lxx genome, and as such increases sensitivity of detection. Primers and probes were purchased from Biosearch Technologies, Inc. The sequences of the primers and probes used in the assays are shown in Table 1 below.

TABLE 1
Oligonucleotide sequences
for TAQMAN ® assays

| TAQMAN ® | | |
|---|---|---|
| assay | Oligo | Sequence (5′->3′) |
| Assay-1 | Forward | CGCCGGATCTGAGACAGTACT |
| | | (SEQ ID NO: 2) |
| | Reverse | ATGTCAATGTTCCACCCATGAG |
| | | (SEQ ID NO: 3) |
| | Probe | FAM-TCACATCGGTACGACTGGGTC |
| | | TCAGC-BHQ1 (SEQ ID NO: 7) |
| Assay-2 | Forward | CCTTGCCAGGCTCATCGT |
| | | (SEQ ID NO: 5) |
| | Reverse | CGGGCGAGACCATAAACATC |
| | | (SEQ ID NO: 6) |
| | Probe | FAM-ATTGGCTGGTCTCCGTCGCAG |
| | | C-BHQ1 (SEQ ID NO: 8) |

Preparation of Copy Number Control DNA Samples for Quantitative Lxx Detection:

Single stranded oligos (DNA sequences corresponding to amplicons for Assay-1 and Assay-2) were used as artificial copy number controls for Lxx. Lxx pathogen copy number is calculated from cycle threshold (Cₜ) values from TAQMAN® assays for Lxx gene and the copy number control. Table 2 below describes the preparation of 1000 μl of a copy number control sample at 1505 copies/μl in TE buffer.

TABLE 2
Copy number control DNA sample preparation

| Reagent | μL |
|---|---|
| Oligo control (1 pM*) | 5 |
| TE | 995 |
| Total volume | 1000 |

*There are 301,000 copies of target in 1 pM oligo stock per μL.

Standard Curve Generation:

1:2, 1:4, 1:8 and 1:16 serial dilutions of the copy number control DNA sample in TE were prepared and a standard curve was generated performing TAQMAN® PCR on the control DNA samples and plotting on the X-axis the values of log₁₀ (copy number) against Cₜ values determined from TAQMAN® PCR of the copy number control DNA sample dilutions.

TAQMAN® Reaction Setup:

Each 10 μl PCR reaction contains 5 μl 2× Sigma JumpStart Master mix (Sigma-Aldrich Corporate), 3 μl diluted sugarcane juice (diluted in TE), 0.2 μl primer and probe (final concentration: 300 nM for primer and 100 nM for probe), 1.8 μl water. Real-time PCR reaction conditions: 95° C., 5 min; 40 cycles at 95° C. 5 sec and 60° C. 30 sec. Real-time PCR was performed in a ABI 7900HT Fast Real-Time PCR System.

Amplicon for TaqMan assay-1 (78 bp):
(SEQ ID NO: 1)
CGCCGGATCTGAGACAGTACTTATCACATCGGTACG
ACTGGGTCTCAGCCGGTCAGCTCATGGGTGGAACAT
TGACAT
Amplicon for TaqMan assay-2 (71 bp):
(SEQ ID NO: 4)
CCTTGCCAGGCTCATCGTCGAGGACCATTGGCTGGT
CTCCGTCGCAGCGAAGATGTTTATGGTCTCGCCCG

Data Analysis:

1) After the PCR was completed, the data was analyzed using the SDS software on the ABI 7900HT Fast Real-Time PCR System

2) Setting of threshold and baselines: The threshold is placed in the region of exponential amplification across all of the amplification plots. The threshold line should be clearly above the background fluorescence and above the level where splitting or fork effects between replicates can be observed. The baseline should be set at a cycle number which is three cycles earlier than the cycle number at which the threshold line crosses the first amplification curve (e.g. earliest Cₜ=24, set the baseline crossing at Cₜ=24−3=21).

3) The Lxx pathogen copy number in sugarcane juice is calculated using the standard curve derived from the Cₜ values of the samples and copy control DNA.

### Results

Blast Analysis of TAQMAN® Assay Specificity:

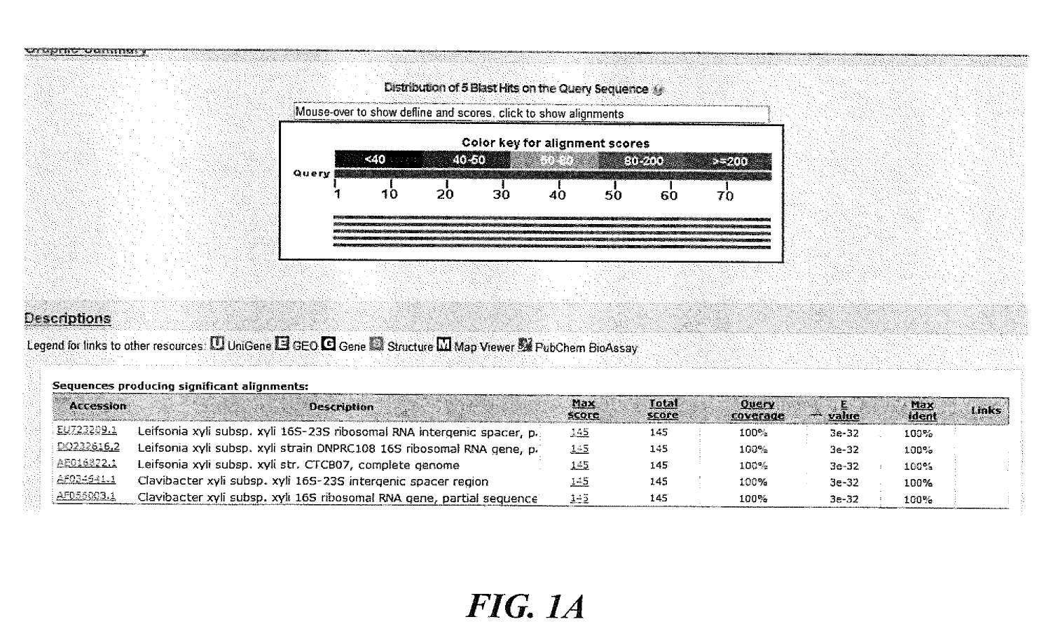

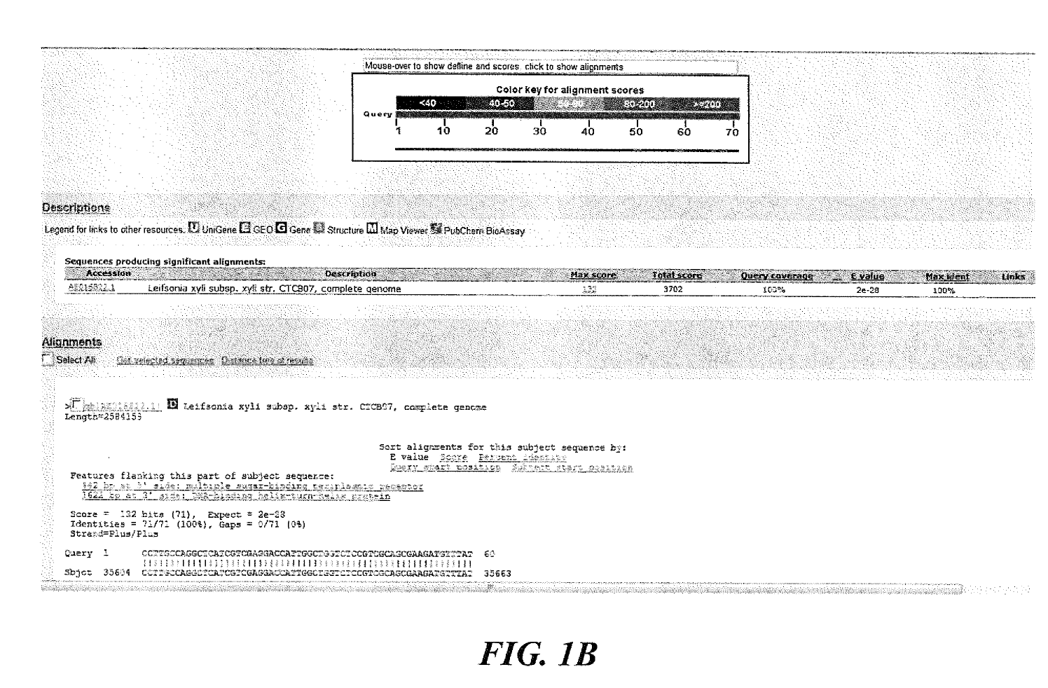

The amplicons for two TAQMAN® assays were subjected to BLAST analysis with GenBank database. The results of this analysis for Assay-1 and Assay 2 are depicted in FIGS. 1A and 1B, respectively. All hits were all from Lxx genomic sequences, and there was no sequence similarity found from other genomes, indicating that the two TAQMAN® assays have high specificity to the Lxx pathogen.

Calculation of Lxx Copy Number:

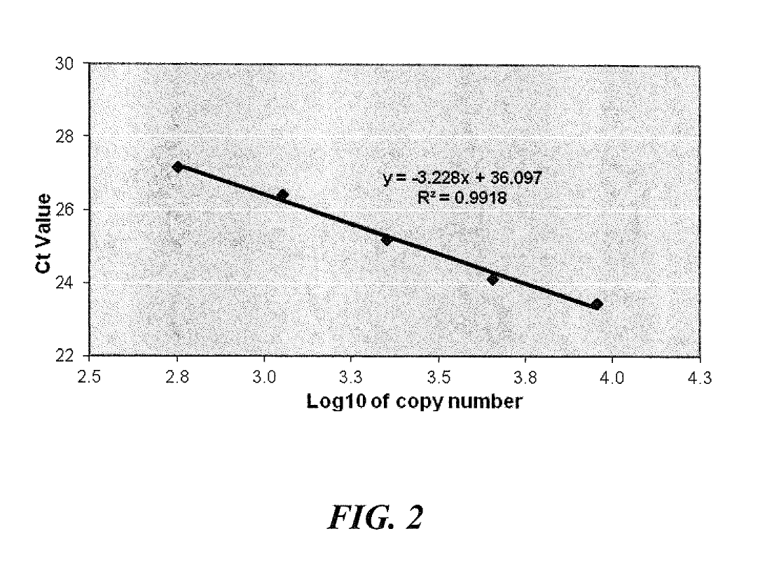

1) Standard curve generation: Values of Log₁₀ (copy control) and Cₜ value (Table 3) were used to generate a standard curve (FIG. 2). The x-axis represents values of log₁₀ (copy number), the y-axis represents Cₜ values (average of three replicates) from TAQMAN® qPCR. The slope was −3.228 from this determination. The slope obtained is an indication of the efficiency of the PCR, wherein for a robust PCR the slope is in the range of −3.1<slope<−3.6.

TABLE 3
Cₜ values determined for standard curve

| Control copy | | | | | |
|---|---|---|---|---|---|
| number | log10 | Ct-1 | Ct-2 | Ct-3 | Mean |
| 4515 | 3.96 | 23.31 | 23.44 | 23.60 | 23.45 |
| 2258 | 3.65 | 24.00 | 24.13 | 24.26 | 24.13 |
| 1129 | 3.35 | 25.06 | 25.23 | 25.32 | 25.20 |
| 564 | 3.05 | 26.12 | 26.69 | 26.41 | 26.41 |
| 282 | 2.75 | 27.05 | 27.16 | 27.29 | 27.17 |

2) Calculation of Lxx copy number in sugarcane juice samples using assay-1 and assay-2. Juice from sugarcane samples infected with Lxx was performed using TAQMAN® qPCR. The results are shown in Tables 4 and 5 for assay-1 and assay-2, respectively.

TABLE 4
Lxx copy number determined in sugarcane
juice sample using assay-1

| | | | | | | Lxx |
|---|---|---|---|---|---|---|
| Sample | Ct-1 | Ct-2 | Ct-3 | Mean | STDEV | copies/mL |
| Cane juice | NA | NA | NA | NA | NA | NA |
| 1:5 dilution | 27.63 | 27.91 | 27.94 | 27.83 | 0.17 | 9.29E+05 |
| 1:10 dilution | 28.88 | 28.95 | 29.17 | 29.00 | 0.16 | 8.13E+05 |
| 1:20 dilution | 30.12 | 29.60 | 30.16 | 29.96 | 0.31 | 8.25E+05 |
| 1:40 dilution | 30.60 | 31.23 | 31.10 | 30.98 | 0.33 | 8.06E+05 |

NA: denotes no PCR amplification signals

TABLE 5
Lxx copy number determined in sugarcane
juice sample using assay-2

| | | | | | | Lxx |
|---|---|---|---|---|---|---|
| Sample | Ct-1 | Ct-2 | Ct-3 | Mean | STDEV | copies/mL |
| Cane juice | 30.43 | 28.04 | 26.64 | 28.37 | 1.92 | 2.97E+03 |
| 1:5 dilution | 22.64 | 22.75 | 23.01 | 22.80 | 0.19 | 8.21E+05 |
| 1:10 dilution | 23.86 | 23.97 | 23.79 | 23.87 | 0.09 | 7.58E+05 |
| 1:20 dilution | 24.95 | 24.94 | 24.76 | 24.88 | 0.11 | 7.31E+05 |
| 1:40 dilution | 26.25 | 26.01 | 25.97 | 26.08 | 0.15 | 6.20E+05 |

When non-diluted sugarcane juice samples were used in the TAQMAN® qPCR assay, no amplification was observed for assay-1, the Cₜ values were relatively high for assay-2. This indicated that the PCR reaction was inhibited by some components in undiluted sugarcane juice. Serial dilutions of sugarcane juice showed that there was no significant PCR inhibition exhibited at a 1:5 dilution. For 1:5 diluted samples, Lxx copy number detected from assay-1 is 9.29×10⁵/mL, and 8.21×10⁵/mL for assay-2. The copy numbers of Lxx pathogen acquired from the two TAQMAN® assays are correlated well. Assay specificity was also tested for, and results showed no PCR amplification in Lxx negative juice samples using either of the two assays.

Assay Sensitivity Test:

Assay sensitivity (LOD) was investigated by using diluted Lxx positive samples. From the Table 6 and 7 below, we found assay-1 quantitatively detected Lxx pathogen at about 12,500 copies/mL. Assay-2 could reliably detect Lxx at about 9,000 copies/mL, with possible qualitative detection of the pathogen as low as about 4,000, copies/mL. There are 26 copies of DNA target for assay-2 in Lxx genome, therefore using assay-2 in detection of Lxx pathogen could increase the limit of detection.

TABLE 6
LOD test of assay-1

| | Cop- | | | | | | | Lxx |
|---|---|---|---|---|---|---|---|---|
| | ies/μL | | | | | | | cop- |
| | (Lxx | | | | | | | ies/μL |
| | con- | | | | | | | by |
| No# | trols) | Ct-1 | Ct-2 | Ct-3 | Mean | STDEV | CV | TaqMan |
| 1 | 1000 | 27.12 | 27.19 | 27.03 | 27.11 | 0.08 | 0.29% | 1012 |
| 2 | 200 | 29.83 | 29.67 | 29.41 | 29.64 | 0.21 | 0.72% | 176 |
| 3 | 100 | 30.58 | 30.35 | 30.46 | 30.46 | 0.11 | 0.37% | 99 |
| 4 | 50 | 31.80 | 31.72 | 32.01 | 31.84 | 0.15 | 0.46% | 38 |
| 5 | 25 | 32.14 | 33.36 | 32.50 | 32.66 | 0.63 | 1.92% | 22 |
| 6 | 12.5 | 33.48 | 32.67 | 33.18 | 33.11 | 0.41 | 1.24% | 16 |
| 7 | 5 | 36.46 | NA | 35.37 | 35.91 | 0.77 | 2.15% | NA |
| 8 | 2.5 | NA | NA | 36.30 | 36.30 | NA | NA | NA |
| 9 | TE | NA | NA | NA | NA | NA | NA | 0 |

TABLE 7
LOD test of assay-2

| | Cop- | | | | | | | Lxx |
|---|---|---|---|---|---|---|---|---|
| | ies/μL | | | | | | | cop- |
| | (Lxx | | | | | | | ies/μL |
| | con- | | | | | | | by |
| No# | trols) | Ct-1 | Ct-2 | Ct-3 | Mean | STDEV | CV | TaqMan |
| 1 | 720 | 22.35 | 22.37 | 22.44 | 22.38 | 0.04 | 0.20% | 692 |
| 2 | 144 | 24.70 | 24.49 | 24.86 | 24.68 | 0.18 | 0.75% | 133 |
| 3 | 72 | 25.34 | 25.75 | 25.64 | 25.58 | 0.21 | 0.82% | 70 |
| 4 | 36 | 26.30 | 26.47 | 26.69 | 26.49 | 0.20 | 0.74% | 37 |
| 5 | 18 | 27.72 | 27.99 | 27.99 | 27.90 | 0.15 | 0.55% | 13 |
| 6 | 9 | 28.64 | 28.48 | 29.40 | 28.84 | 0.49 | 1.70% | 7 |
| 7 | 3.6 | 30.44 | 33.73 | 30.01 | 31.39 | 2.03 | 6.47% | 1 |
| 8 | 1.8 | 31.58 | 29.69 | 33.81 | 31.69 | 2.06 | 6.50% | 1 |
| 9 | TE | NA | NA | NA | NA | NA | NA | 0 |

Comparison of Lxx Titer Between TaqMan and Dot Blot Methods:

24 sugarcane juice samples were tested by both TaqMan and dot blot methods. Results are shown in Table 8 below. Seven samples did not contain Lxx pathogen as undetectable by both methods. Fourteen samples were Lxx positive according to both TaqMan and dot blot. Three samples were Lxx positive as detected only by TaqMan assay, while the dot blot method indicated that the three samples were Lxx negative. Lxx titers for these three samples were relative low, therefore they were beyond the limit of detection using the dot blot detection method. This comparison experiment indicated that the two TaqMan assays are more sensitive than the dot blot method in the detection of the Lxx pathogen.

TABLE 8
Comparison of Lxx titer between TaqMan and dot blot

| | Assay-1 | Assay-2 | Dot blot | |
|---|---|---|---|---|
| | Lxx | Lxx | Lxx | |
| Juice | copies/ | copies/ | copies/ | Comparison TaqMan |
| samples | mL | mL | mL | vs. dot blot |
| 1 | 0 | 0 | 0 | Negative by both methods |
| 2 | 0 | 0 | 0 | Negative by both methods |
| 3 | 0 | 0 | 0 | Negative by both methods |
| 4 | 0 | 0 | 0 | Negative by both methods |
| 5 | 0 | 0 | 0 | Negative by both methods |
| 6 | 0 | 0 | 0 | Negative by both methods |
| 7 | 0 | 0 | 0 | Negative by both methods |
| 8 | 1.2E+07 | 1.1E+07 | 1E+07 | Positive by both methods |
| 9 | 2.6E+07 | 4.0E+07 | 1E+07 | Positive by both methods |
| 10 | 4.6E+07 | 3.7E+07 | 1E+06 | Positive by both methods |
| 11 | 1.5E+07 | 7.1E+06 | 1E+07 | Positive by both methods |
| 12 | 2.1E+06 | 1.6E+06 | 1E+06 | Positive by both methods |
| 13 | 3.7E+06 | 3.3E+06 | 1E+06 | Positive by both methods |
| 14 | 2.4E+06 | 2.0E+06 | 1E+06 | Positive by both methods |
| 15 | 3.6E+06 | 3.7E+06 | 1E+06 | Positive by both methods |
| 16 | 6.1E+06 | 4.9E+06 | 1E+07 | Positive by both methods |
| 17 | 5.3E+06 | 3.9E+06 | 1E+06 | Positive by both methods |
| 18 | 9.7E+06 | 8.9E+06 | 1E+07 | Positive by both methods |
| 19 | 8.0E+06 | 6.3E+06 | 1E+06 | Positive by both methods |
| 20 | 3.8E+07 | 2.1E+07 | 1E+07 | Positive by both methods |
| 21 | 6.1E+06 | 4.9E+06 | 1E+06 | Positive by both methods |
| 22 | 9.4E+04 | 6.1E+04 | 0 | Positive by TaqMan only |
| 23 | 2.8E+04 | 1.7E+04 | 0 | Positive by TaqMan only |
| 24 | 6.5E+05 | 2.7E+05 | 0 | Positive by TaqMan only |

Thus, a rapid diagnostic approach for detection of Lxx pathogen in sugarcane juice based on TAQMAN® qPCR has been provided. This is a quick and/or cost-effective methodology with high sensitivity (LOD is about 4,000 copies/mL). In general this method does not require time-consuming steps of DNA isolation as described in other methods known in the art. Two TAQMAN® assays have been used to detect quantitatively Lxx pathogen from Lxx positive sugarcane juice, and no cross reaction was found from Lxx negative juice. This diagnostic method can be used to detect the presence of ratoon stunting disease in commercial sugarcane, but can also be used as a quality control method in sugarcane nurseries to assist sugarcane breeders in delivering certified ratoon stunting disease-free sugarcane seeds.

The foregoing is illustrative of the present invention, and is not to be construed as limiting thereof. The invention is defined by the following claims, with equivalents of the claims to be included therein.

### REFERENCES

1. Gillaspie A G Jr, Harris R W, Lawson R H. Ratoon stunting disease of sugarcane: isolation of the causal bacterium. Science. 1980, 210 (4476): 1365-1367.
2. Young A J, Petrasovits L A, Croft B J, et al. Genetic uniformity of international isolates of Leifsonia xyli subsp. xyli, causal agent of ratoon stunting disease of sugarcane. Australasian Plant Pathology. 2006, 35 (5): 503-511.
3. Grisham M P, Pan Y, Richard Jr E P. Early detection of Leifsonia xyli subsp. xyli in sugarcane leaves by real-time polymerase chain reaction. Plant Disease. 2007, 91(4): 430-434.
4. Davis M J and Dean J L. Comparison of diagnostic techniques for determining incidence of ratoon stunting disease of sugarcane in Florida. Plant Disease, 1984, 68 (10): 896-899,
5. Gao S J, Pan Y B, Chen R K, et al. Quick detection of Leifsonia xyli subsp. xyli by PCR and nucleotide sequence analysis of PCR amplicons from Chinese Leifsonia xyli subsp, xyli isolates. Sugar Tech, 2008, 10 (4): 334-340.
6. Monteiro-Vitorello C B, Camargo L E, Van Sluys M A, et al. The genome sequence of the gram-positive sugarcane pathogen Leifsonia xyli subsp. xyli. Mol Plant Microbe Interact. 2004, 17 (8): 827-836.

1-24. (canceled)

25. A method for the detection of a microorganism belonging to the genus Leifsonia, comprising:
(a) subjecting said sugar cane sample to quantitative polymerase chain reaction (qPCR) amplification using a pair of oligonucleotide primers, wherein one of the pair of oligonucleotide primers comprises an at least 10 contiguous nucleotide portion identical in sequence to an at least 10 contiguous nucleotide portion of the nucleotide sequence of SEQ ID NO:2, and the other one of the pair of oligonucleotide primers comprises an at least 10 contiguous nucleotide portion identical in sequence to an at least 10 contiguous nucleotide portion of the nucleotide sequence of SEQ ID NO:3 or a complimentary sequence thereto; and
(b) detecting Leifsonia by visualizing the product of the qPCR amplification.

26. The method of claim 25, wherein the pair of oligonucleotide primers consist of the nucleotide sequence of SEQ ID NO:2 and SEQ ID NO:3.

27. The method of claim 25, wherein the microorganism is Leifsonia xyli subsp. xyli, and wherein amplification comprises amplifying at least a part of the Leifsonia xyli subsp. xyli Intergenic Transcribed Spacer (ITS) sequence.

28. The method of claim 25, wherein the amplification comprises either amplifying at least 20 nucleotides of a nucleic acid sequence comprising the sequence of SEQ ID NO:1 or a probe designed from the sequence of SEQ ID NO:1 covalently linked to a fluorescent dye.

29. The method of claim 25, wherein the sugar cane sample is diluted at least five-fold in an aqueous medium prior to subjecting the sample to qPCR.

30. The method of claim 25, wherein the method is carried out in an open system.

31. The method of claim 25, wherein the method is carried out in a closed system.

32. A diagnostic kit used in detecting a microorganism belonging to the genus Leifsonia, comprising the oligonucleotide primers of claim 25.

33. The diagnostic kit of claim 32, wherein the kit further comprises a probe comprising the nucleotide sequence of SEQ ID NO:7.

34. A method for the detection of a microorganism belonging to the genus Leifsonia, comprising:
(a) subjecting a sugar cane sample to quantitative polymerase chain reaction (qPCR) amplification using a pair of oligonucleotide primers, wherein one of the pair of oligonucleotide primers comprises an at least 10 contiguous nucleotide portion identical in sequence to an at least 10 contiguous nucleotide portion of the nucleotide sequence of SEQ ID NO:5, and the other one of the pair of oligonucleotide primers comprises an at least 10 contiguous nucleotide portion identical in sequence to an at least 10 contiguous nucleotide portion of the nucleotide sequence of SEQ ID NO:6; and
(b) detecting Leifsonia by visualizing the product of the qPCR amplification.

35. The method of claim 34, wherein the pair of oligonucleotide primers consist of the nucleotide sequence of SEQ ID NO:5 and SEQ ID NO:6.

36. The method of claim 34, wherein the microorganism is Leifsonia xyli subsp. xyli, and amplification comprises amplifying at least a part of a nucleic acid sequence from ISLxx4 that encodes the tnp transposase from Lxx.

37. The method of claim 34, wherein the amplification comprises either amplifying at least 20 nucleotides of a nucleic acid sequence comprising the nucleotide sequence of SEQ ID NO:4 or a probe designed from the sequence of SEQ ID NO:4 covalently linked to a fluorescent dye.

38. The method of claim 34, wherein the sugar cane sample is diluted at least five-fold in an aqueous medium prior to subjecting the sample to qPCR.

39. The method of claim 34, wherein the method is carried out in an open system.

40. The method of claim 34, wherein the method is carried out in a closed system.

41. A diagnostic kit used in detecting a microorganism belonging to the genus Leifsonia, comprising the pair of primers of claim 34.

42. The diagnostic kit of claim 41, wherein the kit further comprises a probe comprising the nucleotide sequence of SEQ ID NO:8.

43. The diagnostic kit of claim 42, wherein the probe further comprises a fluorescent dye.

44. A method for the detection of the microorganism Leifsonia xyli subsp. xyli, comprising:
(a) obtaining a sugar cane sample;
(b) diluting said sugar cane sample at least five-fold in an aqueous medium;
(c) subjecting a sugar cane sample to quantitative polymerase chain reaction amplification using a pair of oligonucleotide primers and a probe, wherein the pair of oligonucleotide primers is either a first pair or a second pair, wherein the first pair consists of the nucleotide sequence of SEQ ID NO:2 and SEQ ID NO:3 and is used with a probe comprising SEQ ID NO:8, wherein the second pair consists of the nucleotide sequence of SEQ ID NO:5 and SEQ ID NO:6 and is used with a probe comprising SEQ ID NO:9, wherein the amplification comprises amplifying at least part of the nucleic acid comprising the sequence of SEQ ID NO:1 when the first pair is used and the nucleic acid comprising the sequence of SEQ ID NO:4 when the second pair is used; and
(d) detecting Leifsonia xyli subsp. xyli by visualizing the product of the quantitative polymerase chain reaction amplification to determine whether the microorganism is present in said sample.
